(19) United States
(12) Patent Application Publication
(10) Pub. No.: US 2011/0144155 A1
(43) Pub. Date: Jun. 16, 2011

(54) 8-HYDROXYQUINOLINE DERIVATIVES FOR THE TREATMENT OF HEMATOLOGICAL MALIGNANCIES

(76) Inventors: Aaron D. Schimmer, Thornhill (CA); Xiaoming Li, Toronto (CA); Robert Batey, Toronto (CA); Tabitha Wood, Toronto (CA); Xinliang Mao, Markham (CA)

(21) Appl. No.: 12/995,968

(22) PCT Filed: Jun. 5, 2009
(86) PCT No.: PCT/CA09/00776

§ 371 Date: Feb. 15, 2011

(30) Foreign Application Priority Data
Jun. 6, 2008 (US) 61/059489
Nov. 10, 2008 (US) 61/112911

(51) Int. Cl. A61K 31/47; A61P 35/02

(52) U.S. Cl. 514/311

(57) ABSTRACT

The application relates to compositions and methods for treating hematological malignancies and proliferative diseases, disorders and conditions involving increased D-cyclin expression. In particular, the application relates to compositions and methods for treating the hematological malignancies acute myeloid leukemia (AML), lymphoma and multiple myeloma (MM) using 5-nitrogen substituted hydroxy quinolones as well as 7-bromo-5-chloro substituted hydroxy quinolones and 5-chloro substituted hydroxy quinolones.

### FIELD OF THE APPLICATION

This application relates to methods and compositions for the treatment of hematologic malignancies and particularly to methods and compositions for the treatment of acute myeloid leukemia (AML), lymphoma and multiple myeloma (MM) in a subject.

### BACKGROUND OF THE APPLICATION

Acute myeloid leukemia (AML), lymphoma and multiple myeloma (MM) are hematological malignant diseases resulting in the proliferation of abnormal cells of myeloid and lymphoid origin, respectively. Both diseases are characterized by poor responses to standard therapies. For example, elderly patients with either AML, lymphoma or myeloma and poor risk cytogenetics have a median survival of less than one year. Thus, for these patients and those with relapsed refractory disease novel therapies are needed. As many of these patients are frail, therapies that achieve an anti-myeloma or anti-leukemia effect without significant toxicity are highly desirable.

Bortezomib, a proteasome inhibitor, has efficacy in the treatment of myeloma (1) and preliminary data has supported its evaluation for the treatment of other malignancies.

The proteasome mediates the proteasomal degradation pathway which is necessary to rid cells of excess and misfolded proteins as well as to regulate levels of proteins responsible for processes such as cell cycle progression, DNA repair and transcription (reviewed in (2)). The proteasomal degradation pathway is initiated by the sequential activity of E1, E2 and E3 enzymes that mark proteins for degradation by adding chains of ubiquitin molecules to proteins' lysine residues (reviewed in (3, 4)). Once tagged with ubiquitin, proteins are degraded by the 26S proteasome, a multimeric enzymatic complex located in the nucleus and cytoplasm. Inhibition of the proteasome induces cell death through a variety of mechanisms including accumulation of misfolded proteins and NFKB activation (5-8).

The 26S proteasome is comprised of the 19S proteasome that serves a regulatory function and the 20S proteasome that is responsible for the enzymatic degradation of proteins. The 19S proteasome is a multi-subunit complex that recognizes ubiquitin tagged proteins and then de-ubiquitinates, unfolds, and passes them to the 20S proteasome (9). Two 19S subunits cap each end of the barrel-shaped 20S proteasome (10). The 20S proteasome is comprised of alpha and beta subunits that form outer and inner rings of this complex, respectively (11, 12). The alpha subunits on the outside of the 20S proteasome give this complex its barrel shape and allow substrates to enter the center of the barrel (11, 12). The beta subunits form the inside rings of the 20S proteasome and perform the proteolytic function of the complex (12). The 20S proteasome possesses caspase-like, trypsin-like and chymotrypsin-like peptidase activity that is mediated by the β1, β2, and β5 subunits, respectively (11, 13).

A variety of synthetic and natural proteasome inhibitors have been developed and characterized. Proteasome inhibitors such as bortezomib and NPI-0052 bind threonine residues in the active sites of the (3 subunits of the 20S proteasome and thereby inhibit the enzymatic activity of the proteasome (14-16). The FDA-approved proteasome inhibitor bortezomib is a preferential competitive inhibitor of chymotrypsin-like activity which is the rate limiting enzyme in the proteasome (8, 13, 14, 16), whereas, Nereus pharmaceutical's drug NPI-0052 irreversibly inhibits all of the enzymes in the proteasome (8, 14, 15).

Cyclin D2 is over-expressed in multiple myeloma (MM), lymphoma and in high-risk patients with acute myeloid leukemia (AML), contributing to their pathogenesis and chemoresistance (17, 18) (19).

In addition, patients with malignancies including AML have higher levels of copper in their serum compared to healthy controls. Levels of copper are higher in malignant cells compared to normal cells (20-22).

Clioquinol is a copper-binding halogenated 8-hydroxyquinoline that was used in the 1950's to 1970's as an oral anti-parasitic agent for the treatment and prevention of intestinal amebiasis, but its mechanism of action as an anti-microbial was unknown. Clioquinol has recently been shown to inhibit the proteasome in solid tumor cells such as breast and colon cancer cells through a copper-dependent mechanism (23-25).

### SUMMARY OF THE APPLICATION

The application provides a novel treatment for proliferative diseases involving increased expression of D-cyclins and/or hematological malignancies, such as leukemias including acute myeloid leukemia (AML) and acute lymphocytic leukemia (ALL), lymphomas and multiple myeloma (MM) using one or more compounds selected from compounds of Formula I, and pharmaceutically acceptable salts, solvates and prodrugs thereof, a compound of Formula I being:

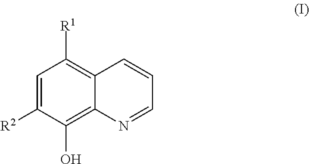

wherein
R¹ is selected from NO₂, NH₂, NH(C₁₋₆alkyl) and N(C₁₋₆alkyl)(C₁₋₆alkyl);
R² is selected from H, halogen, C₁₋₆alkyl, and fluoro-substituted C₁₋₆alkyl,
or

### R¹ is Cl and R² is Br or H.

One aspect of the application is a method of treating a proliferative disease involving increased D-cyclin expression comprising administering to a subject in need of such treatment, an effective amount of one or more compounds selected from a compound of Formula I as defined above, and pharmaceutically acceptable salts, solvates and prodrugs thereof.

Another aspect of the application is a use of an effective amount of one or more compounds selected from a compound of Formula I as defined above, and pharmaceutically acceptable salts, solvates and prodrugs thereof, for the treatment of a proliferative disease involving increased D-cyclin expression.

A further aspect of the application is a use of one or more compounds selected from a compound of Formula I as defined above, and pharmaceutically acceptable salts, solvates and prodrugs thereof, in the preparation of a medicament for the treatment of a proliferative disease involving increased D-cyclin expression.

A further aspect of the application is a method of treating a hematological malignancy comprising administering, to a subject in need of such treatment, an effective amount of one or more compounds selected from a compound of Formula I as defined above, and pharmaceutically acceptable salts, solvates and prodrugs thereof.

Yet another aspect of the application is a use of an effective amount of one or more compounds selected from a compound of Formula I as defined above, and pharmaceutically acceptable salts, solvates and prodrugs thereof, for the treatment of a hematological malignancy.

Another aspect of the application is a use of one or more compounds selected from a compound of Formula I as defined above, and pharmaceutically acceptable salts, solvates and prodrugs thereof, in the preparation of a medicament for treatment of a hematological malignancy.

In certain embodiments the hematological malignancy is leukemia, such as acute myeloid leukemia or acute lymphocytic leukemia, in other embodiments the hematological malignancy is multiple myeloma. In another embodiment, the hematological malignancy is lymphoma. In a further embodiment the lymphoma is Hodgkin's lymphoma. In a further embodiment the lymphoma is low grade non-Hodgkin's lymphoma. In a further embodiment the lymphoma is high grade non-Hodgkin's lymphoma. In a further embodiment, the lymphoma is Hodgkin's lymphoma. In another embodiment, the hematological malignancy expresses increased levels of D-cyclins.

In an embodiment, the compound of Formula I is AHQ. In another embodiment, the compound of Formula I is 8-hydroxy-5-nitroquinoline (HNQ). In a further embodiment, the compound of Formula I is 7-bromo-5-chloro-8-hydroxyquinoline (BCQ). In a further embodiment, the compound of Formula is 5-chloro-8-hydroxyquinoline (COQ).

In an additional aspect of the application, the effective amount of the compound of Formula I, and/or a pharmaceutically acceptable salt, solvate or prodrug thereof, is about 1 to about 200 mg/kg body weight, suitably about 2 to about 100 mg/kg body weight, about 5 to about 50 mg/kg body weight.

In other embodiments, the effective amount of the compound of Formula I, and/or a pharmaceutically acceptable salt, solvate or prodrug thereof, is a daily dose of about 20 to about 5000 mg, about 100 to about 1500 mg or about 400 to about 1200 mg daily dosage. In further embodiments the methods or uses comprise chronic administration, wherein the effective amount of the compound of Formula I and/or a pharmaceutically acceptable salt, solvate or prodrug thereof, is about 20 to about 5000 mg, about 100 to about 1500 mg or about 400 to about 1200 mg and administration or use of the effective amount is one or more times daily for about 1 to about 2 weeks, about 2 to about 4 weeks, and/or more than about 4 weeks.

A further aspect of the application is a pharmaceutical composition for the treatment of proliferative diseases involving increased expression of D-cyclins and/or hematological malignancies comprising one or more compounds selected from a compound of Formula I and pharmaceutically acceptable salts, solvates or prodrugs thereof, and a pharmaceutically acceptable carrier in a dosage form. In a further embodiment, the pharmaceutical composition is formulated for oral administration or injection.

A further aspect of the application is a pharmaceutical composition in solid dosage form comprising about 20 to about 5000 mg of one or more compounds selected from a compound of Formula I as defined above and pharmaceutically acceptable salts, solvates or prodrugs thereof, suitably about 100 to about 1500 mg or about 400 to about 1200 mg of the compound and/or compounds.

A further aspect of the application is a pharmaceutical composition in liquid dosage form comprising about 20 to about 5000 mg of one or more compounds selected from a compound of Formula I as defined above and pharmaceutically acceptable salts, solvates or prodrugs thereof, suitably about 100 to about 1500 mg, or about 400 to about 1200 mg of the compound and/or compounds.

A further aspect of the application is a pharmaceutical composition comprising one or more compounds selected from a compound of Formula I as defined above and pharmaceutically acceptable salts, solvates or prodrugs thereof, and a pharmaceutically acceptable carrier in unit dosage form in an amount suitable to provide about 1 to about 200 mg/kg body weight, suitably about 2 to about 100 mg/kg body weight, or about 5 to about 50 mg/kg body weight of the compound, formulated into a solid oral dosage form, a liquid dosage form, or an injectable dosage form.

A further aspect of the application is a composition formulated as an oral dosage form selected from enteric coated tablets, caplets, gelcaps, and capsules, comprising about 10 to less than about 5000 mg, suitably about 10 to about 1500 mg, or about 30 to about 300 mg of one or more compounds selected from a compound of Formula I as defined above and pharmaceutically acceptable salts, solvates or prodrugs thereof and a pharmaceutically acceptable carrier. Suitably each tablet, caplet, gelcap or capsule comprises about 10 to about 5000 mg, suitably about 10 to about 1500 mg, or about 30 to about 300 mg of the compound and/or compounds.

A further aspect of the application is a commercial package comprising a composition according to the application, and associated therewith instructions for the use thereof for treatment of proliferative diseases involving increased expression of D-cyclins and/or hematological malignancies such as leukemias including acute myeloid leukemia (AML) and acute lymphocytic leukemia (ALL), lymphoma and multiple myeloma (MM).

In other embodiments the methods comprise administering a pharmaceutical composition described herein. In yet other embodiments, the uses comprise use of a pharmaceutical composition described herein.

Other features and advantages of the application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

An embodiment of application will now be described in relation to the drawings in which:

FIG. 1 is a series of graphs demonstrating that 5AHQ reduces the viability of leukemia, lymphoma and myeloma cells. Cell viability of leukemia, lymphoma, myeloma and solid tumor cells forty-eight hours after 5AHQ treatment by MTS assay.

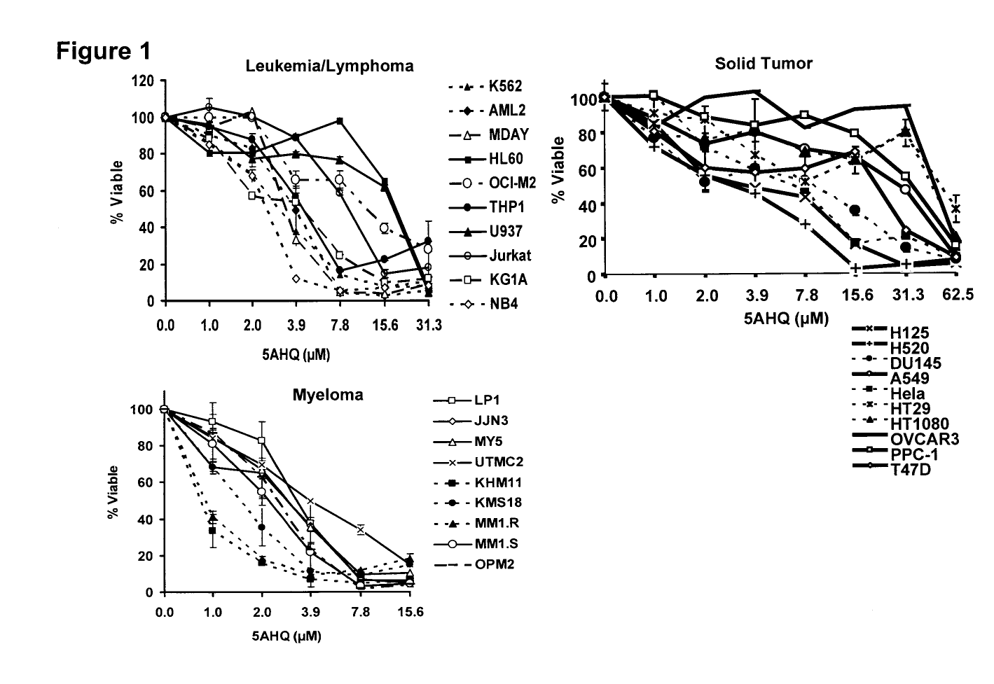

FIG. 2 is a series of graphs demonstrating that HNQ reduces the viability of leukemia, lymphoma and myeloma cells. Cell viability of leukemia, lymphoma, myeloma and solid tumor cells forty-eight hours after HNQ treatment by MTS assay.

FIG. 3 is a series of graphs demonstrating that clioquinol reduces the viability of leukemia, lymphoma and myeloma cells and primary AML patient samples. Cell viability of leukemia, lymphoma, myeloma, solid tumor and primary cells forty-eight hours after clioquinol treatment by MTS assay.

FIG. 4 is a series of graphs that shows treatment with 5AHQ delays tumor growth in mouse models of leukemia and lymphoma.

### DETAILED DESCRIPTION OF THE APPLICATION

### I. Definitions

The term “alkyl” as used herein includes straight and branched chain alkyl groups containing 1, 2, 3, 4, 5 or 6 carbon atoms.

The term “fluoro-substituted” with respect to any specified group as used herein means that the one or more, including all, of the hydrogen atoms in the group have been replaced with a fluorine, and includes trifluoromethyl, pentafluoroethyl, fluoromethyl and the like.

The term “halogen” as used herein means F, Cl, Br or I.

The term “clioquinol” as used herein means 5-chloro-7-iodo-8-hydroxyquinoline.

The term “5AHQ” and/or “AHQ” as used herein means 5-amino-8-hydroxyquinoline and includes all pharmaceutically acceptable salts, solvates, and prodrugs thereof as well as combinations thereof.

The term “HNQ” as used herein means 8-hydroxy-5-nitroquinoline and includes all pharmaceutically acceptable salts, solvates, and prodrugs thereof as well as combinations thereof. HNQ is also known as nitroxoline (NIT), marketed for example under the brand name Nicene.

The term “BCQ” as used herein means 7-bromo-5-chloro-8-hydroxyquinoline and includes all pharmaceutically acceptable salts, solvates, and prodrugs thereof as well as combinations thereof.

The term “COQ” as used herein means 5-chloro-8-hydroxyquinoline and includes all pharmaceutically acceptable salts, solvates, and prodrugs thereof as well as combinations thereof.

The term “compound(s) of the application” as used herein means compound(s) of Formula I, and/or pharmaceutically acceptable salts, solvates and/or prodrugs thereof. It should be noted that the methods and uses extend to cover mixtures of compounds of Formula I and their pharmaceutically acceptable salts, solvates and/or prodrugs.

Potentially, the compounds of the application may have at least one asymmetric centre. Where the compounds described herein possess more than one asymmetric centre, they may exist as diastereomers. It is to be understood that all such isomers and mixtures thereof in any proportion are encompassed within the scope of the present application. It is to be understood that while the stereochemistry of the compounds of the application may be as provided for in any given compound listed herein, such compounds of the application may also contain certain amounts (e.g. less than 20%, suitably less than 10%, more suitably less than 5%) of compounds of the application having alternate stereochemistry.

The term “pharmaceutically acceptable salt” means an acid addition salt, which is suitable for or compatible with the treatment of patients. The term “pharmaceutically acceptable acid addition salt” as used herein means any non-toxic organic or inorganic salt of any base compound of the application. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions.

In general, prodrugs will be functional derivatives of the compounds of the application which are readily convertible in vivo into the compound from which it is notionally derived. Prodrugs of the compounds of the application may be conventional esters formed with the available hydroxy and/or amino group. For example, the available OH and/or NH₂ in the compounds of the application may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the application are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

As used herein, the phrase “effective amount” or “therapeutically effective amount” means an amount effective, at dosages and for periods of time necessary to achieve the desired result. For example in the context or treating a hematological malignancy, an effective amount is an amount that for example induces remission, reduces tumor burden, and/or prevents tumor spread or growth compared to the response obtained without administration of the compound(s). Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

As used herein, to “inhibit” or “suppress” or “reduce” a function or activity, such as proteasomal activity, is to reduce the function or activity when compared to a control, an otherwise same conditions except for a condition or parameter of interest, or alternatively, as compared to another condition. Similarly to “inhibit” or “suppress” or “reduce” expression such as cyclin D expression, is to reduce the level of expression when compared to a control, an otherwise a same condition or parameter or interest, or alternatively as compared to another condition. The terms “inhibitor” and “inhibition”, in the context of the present application, are intended to have a broad meaning and encompass compounds of Formula I which directly or indirectly (e.g., via reactive intermediates, metabolites and the like) act on for example the proteasome, and/or decrease cyclin D expression.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage multiple myeloma can be treated to prevent progression or alternatively a subject in remission can be treated with a compound or composition described herein to prevent recurrence. Treatment methods comprise administering to a subject a therapeutically effective amount of one or more compounds described in the application and optionally consists of a single administration, or alternatively comprises a series of applications. For example, the compounds described herein may be administered at least once a week, about one time per week to about once daily for a given treatment or the compound may be administered one, two, three or four times daily, for example twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

The dosage administered will vary depending on the use and known factors such as the pharmacodynamic characteristics of the particular substance, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

The term “subject” as used herein includes all members of the animal kingdom including mammals, and suitably refers to humans.

The term “hematological malignancy” as used herein refers to cancers that affect blood and bone marrow.

The term “leukemia” as used herein means any disease involving the progressive proliferation of abnormal leukocytes found in hemopoietic tissues, other organs and usually in the blood in increased numbers. For example, leukemia includes acute myeloid leukemia, acute lymphocytic leukemia and chronic myeloma leukemia (CML) in blast crisis.

The term “lymphoma” as used herein means any disease involving the progressive proliferation of abnormal lymphoid cells. For example, lymphoma includes Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas (30). Non-Hodgkin's lymphomas can also for example be as classified using the WHO and REAL classification (30).

The term “myeloma” and/or “multiple myeloma” as used herein means any tumor or cancer composed of cells derived from the hemopoietic tissues of the bone marrow. Multiple myeloma is also knows as MM and/or plasma cell myeloma.

The term “proliferative disease involving increased expression of cyclin D” means any disease where a cell type increases in numbers and has increased expression of cyclin D. Three D-cyclins are known including cyclin D1, cyclin D2 and cyclin D3. For example, the cyclin D that has increased expression is cyclin D2. One skilled in the art would readily understand that cyclin D expression is easily detected by methodologies known in the art such as protein detection methods such as immunoblotting and ELISA and nucleic acid methods such as RT-PCR and northern analysis. Increased cyclin D expression can be determined by comparing the level of cyclin D expression to one or more control samples, individually or pooled.

As used herein, “contemporaneous administration” and “administered contemporaneously” means that two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

The term “cell death” as used herein includes all forms of cell death including necrosis and apoptosis.

The “proteasome” as used herein refers to a multimeric enzymatic complex involved in the degradation of protein. The proteasome comprises multiple protease activities including a chymotrypsin-like protease activity. As mentioned, the proteasomal degradation pathway rids cells of excess and misfolded proteins as well as to regulate levels of proteins responsible for processes such as cell cycle progression, DNA repair and transcription (reviewed in (2)).

The term “proteasomal activity” as used herein refers to an activity of the proteasome and “chymotrypsin-like proteasomal activity” refers to the protease activity of the proteasome that is specific for chymotrypsin or chymotrypsin-like substrates.

The term “pharmaceutically acceptable” means compatible with the treatment of animals, in particular, humans.

### II. Method/Uses

Novel therapeutics for treating hematological malignancies such as multiple myeloma (MM), lymphoma acute lymphoid leukemia (ALL) and acute myeloid leukemia (AML) have been identified. Using a chemical biology screen for inhibitors of cyclin D2 transactivation, it has surprisingly been found that 5-nitrogen substituted and other analogs of the anti-parasitic compound clioquinol are inhibitors of cyclin D2 transactivation. Furthermore, it has been shown that these compounds reduce the viability of leukemia, lymphoma and myeloma cells and delay tumor growth.

Accordingly, the application describes a method of treating hematological malignancies including leukemia, lymphoma and multiple myeloma comprising administering an effective amount of one or more compounds selected from a compound of Formula I, and pharmaceutically acceptable salts, solvates and prodrugs thereof to a subject in need of such a treatment wherein the compound of Formula I is:

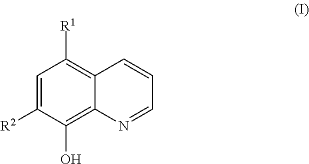

wherein
R¹ is selected from NO₂, NH₂, NH(C₁₋₆alkyl) and N(C₁₋₆alkyl)(C₁₋₆alkyl);
R² is selected from H, halogen, C₁₋₆alkyl, and fluoro-substituted C₁₋₆alkyl,
or

### R¹ is Cl and R² is Br or H.

Also disclosed herein is a use of one or more compounds selected from a compound of Formula I as defined above, and pharmaceutically acceptable salts, solvates and prodrugs thereof for the treatment of a hematological malignancy such as a leukemia, lymphoma or multiple myeloma.

Further described herein is a use of one or more compounds selected from a compound of Formula I, as defined above, and pharmaceutically acceptable salts, solvates and prodrugs thereof in the preparation of a medicament for the treatment of a hematological malignancy such as leukemia, lymphoma and/or multiple myeloma.

In the methods and uses described in the application, the compounds of Formula I include those in which R¹ is selected from NO₂, NH₂, NH(C₁₋₆alkyl) and N(C₁₋₆alkyl)(C₁₋₆alkyl). In an embodiment of the application R¹ is selected from NO₂, NH₂, NH(C₁₋₄alkyl) and N(C₁₋₄alkyl)(C₁₋₄alkyl). In a further embodiment of the application, R¹ is selected from NO₂, NH₂, NHCH₃ and N(CH₃)₂. In a yet another embodiment, R¹ is selected from NO₂ and NH₂.

In the methods and uses of the application, the compounds of Formula I include those in which R² is selected from H, halogen, C₁₋₆alkyl, and fluoro-substituted C₁₋₆alkyl. In an embodiment of the application, R² is selected from H, Cl, F, I, C₁₋₄alkyl, and fluoro-substituted C₁₋₄alkyl. In another embodiment of the disclosure R² is selected from H, Cl, I, CH₃, and CF₃. In yet another embodiment of the application, R² is H.

AHQ, HNQ, BCQ and COQ have been shown herein to share the common activity of to reducing the viability of several leukemia and lymphoma cells including, primary leukemia and lymphoma cells. AHQ has been further shown herein to reduce tumor burden in a well studied mouse model of leukemia (31, 32). Accordingly it is clear from the results reported herein that the compounds of Formula I will have in vivo efficacy in the treatment of hematological malignancies.

It is an embodiment that, in the methods and uses described herein, the compounds of Formula I are selected from one or more of AHQ, HNQ, BCQ and COQ and pharmaceutically acceptable salts, solvates and prodrugs thereof.

In an embodiment, the hematological malignancy is a leukemia such as acute myeloid leukemia. In another embodiment, the leukemia is acute lymphocytic leukemia. In another embodiment the subject has high-risk acute myeloid leukemia. In another embodiment, the hematological malignancy is multiple myeloma. In another embodiment, the hematological malignancy is lymphoma. In a further embodiment, the lymphoma is lymphoma is Non-Hodgkin's lymphoma. In another embodiment, the lymphoma is Hodgkin's lymphoma. In a further embodiment, the Non-Hodgkin's lymphoma is selected from indolent and aggressive Non-Hodgkin's lymphoma. In another embodiment, the aggressive Non-Hodgkin's lymphoma is selected from an intermediate and a high grade lymphoma. In another embodiment, the indolent Non-Hodgkin's lymphoma is a low grade lymphoma.

It has also been demonstrated that the compounds of Formula I induce cell death in leukemia, lymphoma and multiple myeloma cells. Accordingly, the application includes a method of inducing cell death in a leukemia cell, a lymphoma cell and/or a myeloma cell comprising administering an effective amount of one or more compounds selected from a compound of Formula I as defined above, and pharmaceutically acceptable salts, solvates and prodrugs thereof, to the cell.

In another aspect, the application describes treating a proliferative disease involving increased D-cyclin expression. In one embodiment, the method of treating a proliferative disease involving increased D-cyclin expression comprises, administering to a subject in need of such treatment, an effective amount of one or more compounds selected from a compound of Formula I as defined above, and pharmaceutically acceptable salts, solvate and prodrugs thereof.

Another aspect of the application is a use of an effective amount of one or more compounds selected from a compound of Formula I as defined above, and pharmaceutically acceptable salts, solvates and prodrugs thereof, for the treatment of a proliferative disease involving increased D-cyclin expression.

A further aspect of the application is a use of compound selected from a compound of Formula I as defined above, and pharmaceutically acceptable salts, solvates and prodrugs thereof, in the preparation of a medicament for the treatment of a proliferative disease involving increased D-cyclin expression.

In one embodiment, the increased D-cyclin expression comprises, increased cyclin D2 expression.

In another aspect, the application also includes a method of inhibiting cyclin D expression in a cell or in a subject, comprising administering an effective amount of one or more compounds selected from a compound of Formula I, as defined above and pharmaceutically acceptable salts, solvates and prodrugs thereof to the cell or the subject in need of such a treatment.

Inhibiting cyclin D expression means in one embodiment, reducing expression by at least 50%, at least 60%, at least 70%, at least 80%, at least 90% or at least 95% as determined using assays known in the art, for example immunoblotting.

In one embodiment, the subject in need thereof has leukemia. In another embodiment, the subject has acute myeloid leukemia. In another embodiment, the subject has high-risk acute myeloid leukemia. In another embodiment, the subject has multiple myeloma. In another embodiment, the subject has lymphoma. In one embodiment, the lymphoma is Non-Hodgkin's lymphoma. In another embodiment, the lymphoma is Hodgkin's lymphoma. In a further embodiment, the Non-Hodgkin's lymphoma is selected from indolent and aggressive Non-Hodgkin's lymphoma. In another embodiment, the aggressive Non-Hodgkin's lymphoma is selected from an intermediate and a high grade lymphoma. In another embodiment, the indolent Non-Hodgkin's lymphoma is a low grade lymphoma. In a further embodiment, the subject has a refractory malignancy. In another embodiment, the subject in need thereof has a proliferative disorder with increased cyclin D expression.

In another aspect, the application provides methods and uses comprising identifying a subject in need thereof, wherein the subject has a proliferative disorder and/or hematological malignancy with increased cyclin D expression, and administering an effective amount of one or more compounds selected from a compound of Formula I as defined above, and pharmaceutically acceptable salts, solvate and prodrugs thereof.

Also included are methods and uses for treating a proliferative disease involving increased cyclin D expression and/or methods and uses for treating a hematological malignancy such as leukemia, lymphoma or MM such as the methods and uses described herein, comprising administering an effective amount of one of the pharmaceutical compositions described herein to a subject in need of such treatment. In a further embodiment, the methods and uses comprise administering a formulation and/or dosages described herein.

It has also been found that a copper compound can enhance the therapeutic effect the compounds of Formula I. Accordingly in certain aspects, the methods comprise contemporaneous administration of one or more compounds selected from a compound of Formula I as defined above, and pharmaceutically acceptable salts, solvates and prodrugs thereof, and a copper compound. In one embodiment the copper compound is a copper salt. In another embodiment the copper compound is copper oxide. In certain embodiments the copper compound is administered, contemporaneously as an oral tablet or capsule.

Without wishing to be bound by theory, the mechanism of action of the compounds of Formula I may involve one or more of the following pathways. As mentioned, D-cyclins are over-expressed in multiple myeloma (MM), lymphoma and a subset of high-risk patients with acute myeloid leukemia (AML), contributing to their pathogenesis and chemoresistance (17, 18) (19). Further inhibition of the proteosome by Bortezomib, a protease inhibitor has been shown to have efficacy in the treatment of multiple myeloma. The proteasomal degradation pathway is necessary to rid cells of excess and misfolded proteins as well as regulate levels of proteins responsible for processes such as cell cycle progression, DNA repair and transcription (reviewed in (2)). In addition, patients with malignancies including AML have higher levels of copper in their serum. As it has been shown that compounds such as AHQ, HNQ, BCQ and COQ inhibit the proteasome and also bind copper, their ability to inhibit the proteasome may be partly related to their ability to bind copper. However, they also likely have copper-independent effects on the proteasome. Therefore, these compounds may be affecting one or more of these pathways.

### III. Compositions

The application also describes compositions comprising the compounds of the application for the treatment of hematological malignancies and/or proliferative disorders involving increased cyclin D expression.

Accordingly, a further aspect of the application is a pharmaceutical composition for the treatment of proliferative diseases involving increased expression of D-cyclins and/or hematological malignancies comprising one or more compounds selected from a compound of Formula I and pharmaceutically acceptable salts, solvates and prodrugs thereof, and a pharmaceutically acceptable carrier in a dosage form, wherein the compound of Formula I is:

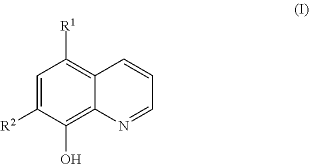

wherein
R¹ is selected from NO₂, NH₂, NH(C₁₋₆alkyl) and N(C₁₋₆alkyl)(C₁₋₆alkyl);
R² is selected from H, halogen, C₁₋₆alkyl, and fluoro-substituted C₁₋₆alkyl
or

R¹ is Cl and R² is Br or H.

In the compositions the application, the compounds of Formula I include those in which R¹ is selected from NO₂, NH₂, NH(C₁₋₆alkyl) and N(C₁₋₆alkyl)(C₁₋₆alkyl). In an embodiment of the application R¹ is selected from NO₂, NH₂, NH(C₁₋₄alkyl) and N(C₁₋₄alkyl)(C₁₋₄alkyl). In a further embodiment of the application, R¹ is selected from NO₂, NH₂, NHCH₃ and N(CH₃)₂. In a yet another embodiment, R¹ is selected from NO₂ and NH₂.

In the compositions of the application, the compounds of Formula I include those in which R² is selected from H, halogen, C₁₋₆alkyl, and fluoro-substituted C₁₋₆alkyl. In an embodiment of the application R² is selected from H, Cl, F, I, C₁₋₄alkyl, and fluoro-substituted C₁₋₄alkyl. In another embodiment of the disclosure R² is selected from H, Cl, I, CH₃, and CF₃. In yet another embodiment of the application, R² is H.

It is an embodiment that, in the compositions of the application, the compounds of Formula I are selected from one or more of AHQ, HNQ, BCQ and COQ, and pharmaceutically acceptable salts, solvates and prodrugs thereof.

The compounds of the application are suitably formulated into pharmaceutical compositions for administration to human subjects in a biologically compatible form suitable for administration in vivo.

The application in one aspect, also describes a pharmaceutical composition comprising an effective amount of one or more compounds of the application and a pharmaceutically acceptable carrier for treatment of a leukemia, lymphoma and/or multiple myeloma in a subject in need of such treatment.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

In an embodiment of the application the pharmaceutical composition contains about 0.01% to about 1%, suitably about 0.01% to about 0.5%, of one or more compounds of the application. The composition may be prepared, for example, by mixing the carrier and the compound(s) at a temperature of about 40° C. to about 70° C.

Suitable vehicles are described, for example, in Remington's Pharmaceutical Sciences (2003—20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The dosage form may be solid or liquid.

Accordingly, a further aspect of the application is a composition formulated for as an oral dosage form selected from enteric coated tablets, caplets, gelcaps, and capsules, each unit dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from a compound of Formula I as defined above and a pharmaceutically acceptable salt, solvate or prodrug thereof and a pharmaceutically acceptable carrier.

In one embodiment, the application describes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, immediate release and timed-release formulations. Examples of timed-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. It is also possible to freeze-dry the compounds of the application and use the lyophilizates obtained, for example, for the preparation of products for injection.

Accordingly, a further aspect of the application is a pharmaceutical composition in solid dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from a compound of Formula I as defined above and a pharmaceutically acceptable salt, solvate or prodrug thereof and a pharmaceutically acceptable carrier.

Compositions suitable for buccal or sublingual administration include tablets, lozenges, and pastilles, wherein the active ingredient is formulated with a carrier such as sugar, acacia, tragacanth, and/or gelatin and/or glycerin.

In another embodiment, the application describes a pharmaceutical composition wherein the dosage form is a liquid dosage form. A liquid dosage form is to be understood to refer to non-solid dosage forms suitable for, but not limited to, intravenous, subcutaneous, intramuscular, or intraperitoneal administration. Solutions of compounds of Formula I described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999.

Accordingly, a further aspect of the application is a pharmaceutical composition in liquid dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from a compound of Formula I as defined above and pharmaceutically acceptable salts, solvates or prodrugs thereof and a pharmaceutically acceptable carrier.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In one embodiment the dosage form comprises about 10 mg to about 5000 mg of a compound of the application. In another embodiment, the dosage form comprises about 10 mg to about 1500 mg of a compound of the application. In yet another embodiment, the dosage form comprises about 30 mg to about 300 mg of a compound of the application. In other embodiments, the dosage form comprises about 10 to about 3500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of a compound of the application.

In one embodiment the dosage, for example the daily dosage, comprises about 20 mg to about 5000 mg of a compound of the application. In another embodiment, the dosage comprises about 100 mg to about 1500 mg of a compound of the application. In yet another embodiment, the dosage comprises about 400 to about 1200 mg of a compound of the application. In other embodiments, the dosage form comprises about 10 to about 3500 mg, about, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg of a compound of the application.

The dosage form may alternatively comprise about 1 to about 200 mg of a compound of the application/kg body weight, about 2 to about 100 mg of a compound of the application/kg body weight, about 20 to about 100 mg of a compound of the application/kg body weight, about 5 to about 50 mg of a compound of the application/kg body weight about 5 to about 25 mg of a compound of the application/kg body weight, or about 5 to about 15 mg of a compound of the application/kg body weight of a subject in need of such treatment formulated into a solid oral dosage form, a liquid dosage form, or an injectable dosage form. In one embodiment, the dosage comprises about 5 to about 15 mg of a compound of the application kg body weight of a subject in need of such treatment formulated into a solid oral dosage form, a liquid dosage form, or an injectable dosage form.

The dosage form comprises an effective amount or a therapeutically effective amount. In one embodiment the dosage form comprises about 10 to about 5000 mg of a compound of the application. In another embodiment, the dosage form comprises about 10 to about 1500 mg of the compound. In another embodiment, the dosage form comprises about 30 to about 300 mg of the compound.

As mentioned above, also included are methods and uses of treating a proliferative disease involving increased cyclin D expression or a hematological malignancy such as AML or MM and administering an effective amount of one of the pharmaceutical compositions described herein to a subject in need of such treatment.

Accordingly, a further aspect of the application is a composition, wherein the amount of the compound of Formula I, and/or pharmaceutically acceptable salt, solvate and/or prodrug thereof, is an effective amount for treatment of a haematological malignancy, in one embodiment, leukaemia such as acute myeloid leukemia, lymphoma or multiple myeloma.

Another aspect provides a commercial package comprising a composition described herein, and associated therewith instructions for the use thereof for treatment of a hematological malignancy such as leukemia including acute myeloid leukemia, lymphoma or multiple myeloma or a proliferative disease, disorder or condition involving increased cyclin D such as cyclin D2, expression in a subject in need of such treatment. In another embodiment, a commercial package is provided comprising a composition described herein, and associated therewith instructions for the use thereof for inhibiting cyclin D expression. Another embodiment provides a commercial package comprising a composition described herein, and associated therewith instructions for the inducing cell death in a multiple myeloma, lymphoma or a leukemia cell.

The following non-limiting examples are illustrative of the present application:

### EXAMPLES

### Example 1

### Materials and Methods

### Cell Culture, Constructs and Transduction

Mouse fibroblast NIH3T3 cells were maintained in Dulbeco's Modified Eagle's medium plus 10% calf serum (Hyclone, Logan, Utah). Myeloma (LP1, JJN3, MY5, UTMC2, KHM11, KMS18, MM1.R, MM1.S, OPM2) leukemia (K562, AML2, HL60, OCI-M2, THP1, Jurkat, KG1A, NB4, U937), and lymphoma (MDAY-D2), cell lines were grown in Roswell Park Memorial Institute (RPMI) 1640 media. Solid tumor (H125, H520, DU145, A549, Hela, HT29, HT1080, OVCAR3, PPC-1, and T47D) were grow in RPMI-1640 plus 10% fetal bovine serum (FBS) (Hyclone, Logan, Utah). All the media were supplemented with 1 mM glutamate and antibiotics. Cells were cultured at 37° C. with 5% CO₂ in a humid incubator. Full-length c-maf cDNA was subcloned into an IRES-GFP-MIEV retroviral vector. NIH3T3 cells were infected with this construct and stable cells expressing GFP and c-maf were selected by flow cytometry and immunoblotting, respectively. The full-length c-maf was also subcloned into a pcDNA3.1 vector under the control of a CMV promoter.

The promoter of cyclin D2 (−894 to −4), containing c-maf responsive element sequence (MARE), was cloned from HeLa cell genomic DNA and subcloned into the pGL2 luciferase reporter vector (Promega, Madison, Wis.). This construct was co-transfected with pcDNA3.1 containing a neomycin resistance gene into NIH3T3 wild type cells and NIH3T3 cells stably over-expressing c-maf-IRES-GFP. Cells stably expressing c-maf, GFP, and luciferase were selected for further application.

### High Throughput Screen for Inhibitors of Cyclin D2 Transactivation

NIH3T3 cells stably expressing c-maf and the cyclin D2 promoter driving luciferase (13,000 cells per well) were plated in 96-well plates by the Biomek FX liquid handler (Beckman, Fullerton, Calif.). The same workstation was used for plate formatting and reagent distribution. After the cells had adhered (6 hr after plating), they were treated with aliquots of molecules from LOPAC (Sigma, St. Louis, Mo.) and Prestwick (Prestwick Chemical Inc, Illkirch, France) libraries. Final concentration of LOPAC compounds was 5 μM (0.05% DMSO) while for the Prestwick library, 10 ng of each sample was added, resulting in an average final concentration of approximately 5 μM (0.1% DMSO). Control wells, treated with vehicle alone containing consistent levels of DMSO, were distributed in the first and last columns of the plate to monitor signal variability. Cells were incubated with the molecules at 37° C. for 20 hours. After incubation, cyclin D2 transactivation was assessed by the luciferase assay and viability was assessed by the MTS assay.

### Luciferase Assay

Luciferase activity was assessed according to the manufacturer's instructions (Promega, Madison, Wis.). Briefly, the cell culture medium was removed using an EMBLA plate washer (Molecular Devices, Sunnyvale, Calif.) and 1× Glo Lysis buffer (Promega) was added by the robotic liquid handler. After 10 min incubation, an equal volume of Bright-Glo Luciferase substrate (Promega) was added and the luminescence signal was detected with a 96-well Luminoskan luminescence plate reader (Thermo Labsystem, Waltham, Mass.) with a 5 second integration.

### Cell Viability

Cell viability was assessed with the CellTiter96® Aqueous Non-Radioactive Assay kit according to manufactures' instructions (Promega). Apoptosis was measured by flow cytometry to detect cell surface Annexin V expression and propidium iodide (PI) uptake (Biovision, Mountain View, Calif., USA) as previously described (26).

### Immunoblotting

To prepare cytosolic extracts, NIH3T3 cells and myeloma cells were washed with phosphate-buffered saline (PBS, pH 7.4) and suspending in lysis buffer [10 mM Tris (pH 7.4), 150 mM NaCl, 0.1% Triton X-100, 0.5% sodium deoxycholate, and 5 mM EDTA] containing protease inhibitors (Complete tablets, Roche, Indianapolis, Ind.). Protein concentrations were determined by the Bradford assay. Immunoblot assays were performed by subjecting equal amounts of protein to SDS-PAGE gels followed by transfer to Nitrocellulose membranes. Membranes were probed with polyclonal rabbit anti-human cyclin D2 (0.5 μg/ml, both from Santa Cruz Biotech, Santa Cruz, Calif.), or monoclonal mouse-anti human p21 (1:200 v/v, Santa Cruz Biotech), monoclonal mouse-anti human p27 (1:2,500 v/v BD Transduction Laboratories), polyclonal mouse-anti human ubiquitin (1:2,000 v/v Calbiochem) and monoclonal mouse-anti-β-actin (1:10,000 v/v) (Sigma, St. Lois, Mo.). Secondary antibodies (Amersham Bioscience UK, Little Chalfont, England) were horseradish peroxidase (HRP)-conjugated goat anti-mouse IgG (1:10,000 v/v) and anti-rabbit (1:5,000 v/v). Detection was performed by the Enhanced Chemical Luminescence (ECL) method (Pierce, Rockford, Ill.).

### Cell Cycle Analysis

Cells were harvested, washed with cold PBS, suspended in 70% cold ethanol and incubated overnight at −20° C. Cells were then treated with 100 ng/ml of DNase-free RNase (InvitroGen) at 37° C. for 30 min, washed with cold PBS, and resuspended in PBS with 50 μg/ml of propidium iodine. DNA content was analyzed by flow cytometry (FACSCalibur, Becton Dickinson, Florida, USA). The percentage of cells in each phase of the cell cycle was calculated with ModiFit software (Becton Dickinson).

### Proteasome Activity

Cellular proteins were extracted from cells with lysis buffer (50 mM HEPES pH 7.5, 150 mM NaCl, 2 mM ATP, and 1% Triton X-100). The chymotrypsin-like activity of the proteasome was measured by incubating equal amounts of protein with the preferential chymotrypsin-like substrate Suc-LLVY-AMC in assay buffer (50 mM Tris-HCl, pH 7.5, 150 mM NaCl) for two hours. After incubation, was added and the rate of free AMC was measured over time with a florescent spectrophotometric plate reader (excitation=380 nm, emission=460 nm).

### In Vivo Studies

DBA-2 mice were injected intraperitoneally with MDAY-D2 murine leukemia cells. Mice were then treated twice daily by oral gavage with clioquinol (100 mg/kg) dissolved in intralipid or intralipid control. Ten days after treatment, mice were sacrificed, the intraperitoneal tumor excised, and the weight and volume of the tumor measured.

Sublethally irradiated (3.54 Gy) NOD/SCID mice were injected subcutaneously with K562 human leukemia cells. One week later, mice were then treated daily by oral gavage with 5AHQ (50 mg/kg) in water. Three weeks after injection of cells, mice were sacrificed, the subcutaneous tumor excised, and the weight and volume of the tumor measured.

A High Throughput Screen Identifies c-maf Dependent and Independent Inhibitors of the Cyclin D2 Promoter

Cyclin D2 is over-expressed in multiple myeloma (MM) and a subset of high-risk patients with acute myeloid leukemia (AML), contributing to their pathogenesis and chemoresistance (17, 18) (19). One of the regulators of cyclin D2 is the oncogene c-maf that is also frequently over-expressed in MM (27). Therefore, it was sought to identify c-maf dependent and independent inhibitors of cyclin D2. To identify such small molecule inhibitors, a high throughput chemical genomics screen was developed. NIH 3T3 cells stably over-expressing a c-maf-IRES-GFP cassette in an MIEV vector and the cyclin D2 promoter (−894 bp to −4 bp) driving firefly luciferase were seeded in 96 well plates and treated with aliquots of the LOPAC (1280 compounds) and Prestwick (1120 compounds) libraries of off-patent drugs and chemicals. Compounds were tested at a final concentration of ˜5 μM in <0.01% DMSO. Sixteen hours after the addition of the compounds, luciferase expression was measured as a marker of cyclin D2 transactivation. From this screen, both c-maf dependent (28) and c-maf independent inhibitors of the cyclin D2 promoter were identified. The latter included the antimicrobial agent clioquinol. Additional studies demonstrated that clioquinol reduced levels of cyclin D2 in myeloma and leukemia cells, arrested cells in the G1 phase and increased expression of p21 and p27 at low micromolar concentrations.

### Compounds of Formula I Induce Cell Death in Leukemia and Myeloma Cell Lines

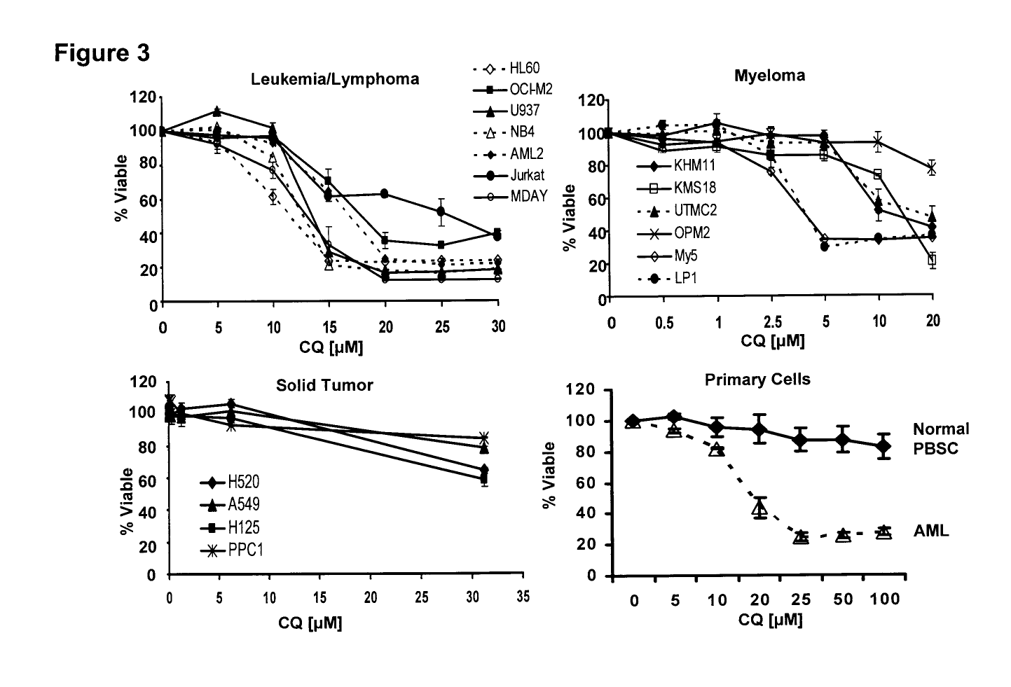

The effects of clioquinol on the viability of acute leukemia/lymphoma (n=7), myeloma (n=14), and solid tumor (n=5) cell lines as well as primary AML samples (n=6) and primary normal hematopoietic cells (n=3) was tested. Forty-eight hours after incubation, clioquinol induced cell death in 6/7 AML, 12/14 myeloma, 0/5 solid tumor, 6/7 primary AML patient samples and 0/3 normal hematopoietic cells with an IC₅₀<20 μM (FIG. 3). Of note, after oral administration of clioquinol, trough serum concentrations of 20 μM can be achieved in patients (29). Reductions in cell viability were associated with clioquinol's ability to inhibit the proteasome, and no proteasomal inhibition was detected in clioquinol-resistant cells, including the normal hematopoietic cells.

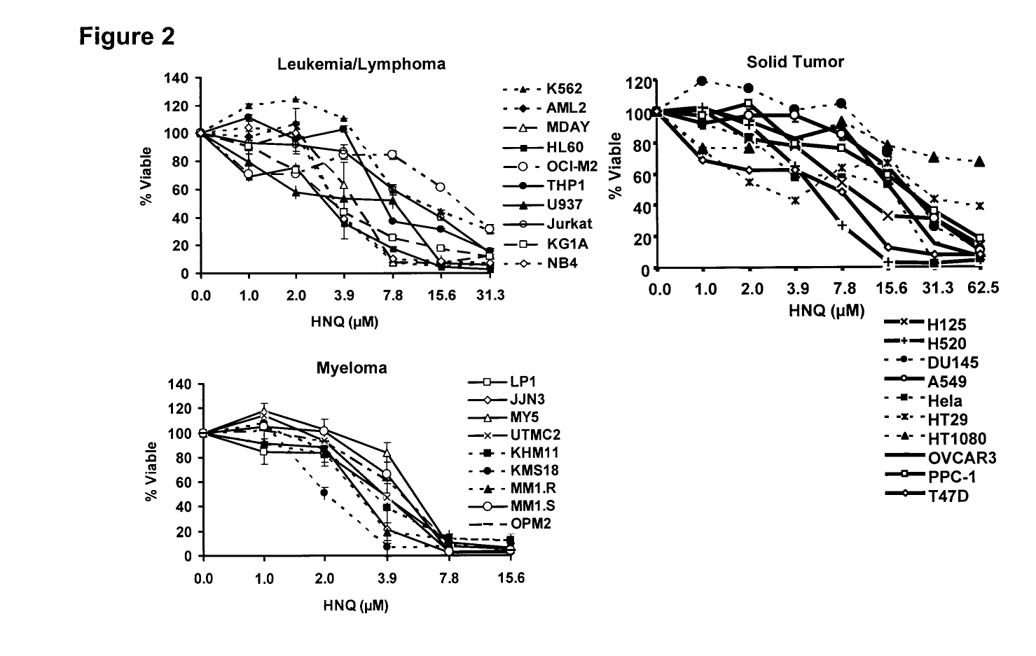

The effects of 5AHQ on the viability of acute leukemia/lymphoma (n=10), myeloma (n=9), and solid tumor (n=10) cell lines was tested. Forty-eight hours after incubation, 5AHQ induced cell death in 7/10 AML, 9/9 myeloma, and 3/10 solid tumor, with an IC₅₀<15 μM (FIG. 2). Reductions in cell viability were associated with 5AHQ's ability to inhibit the proteasome, and no proteasomal inhibition was detected in 5AHQ-resistant cells. The effects of other compounds of Formula I on the viability of AML (K562 and OCI AML2), lymphoid leukemia/lymphoma (MDAY-D2), myeloma (LP1) and solid tumor (A549 lung cancer) cells lines are also shown (FIG. 3). These compounds reduced cell viability at low micromolar concentrations. Cell death induced by these compound was associated with inhibition of the proteasome. Unlike clioquinol, 5AHQ lacks a halogen at R1 and comprises instead a NH2 group. Compounds lacking a halogen at this position were as, or more effective, or potent at inducing cancer cell death. The increased solubility and/or potency of the analogues would not be expected based on structure.

Cell death induced by the compounds was copper dependent as supplementing copper in the medium enhanced the toxicity of these compounds and increased their ability to inhibit the proteasome. Copper binding alone was not sufficient to inhibit the proteasome as the strong copper binding compounds tetrathiomoylbdate did not inhibit the proteasome nor induce cell death. Moreover, compounds of the application that bound copper but did not inhibit the proteasome also did not induce cell death.

Thus, these results indicate that the compounds induce cell death through a copper-dependent mechanism and are consistent with its effects as a copper dependent proteasome inhibitor.

### The Compounds of the Application Delay Tumor Growth in a Xenograft Model of Leukemia and Lymphoma

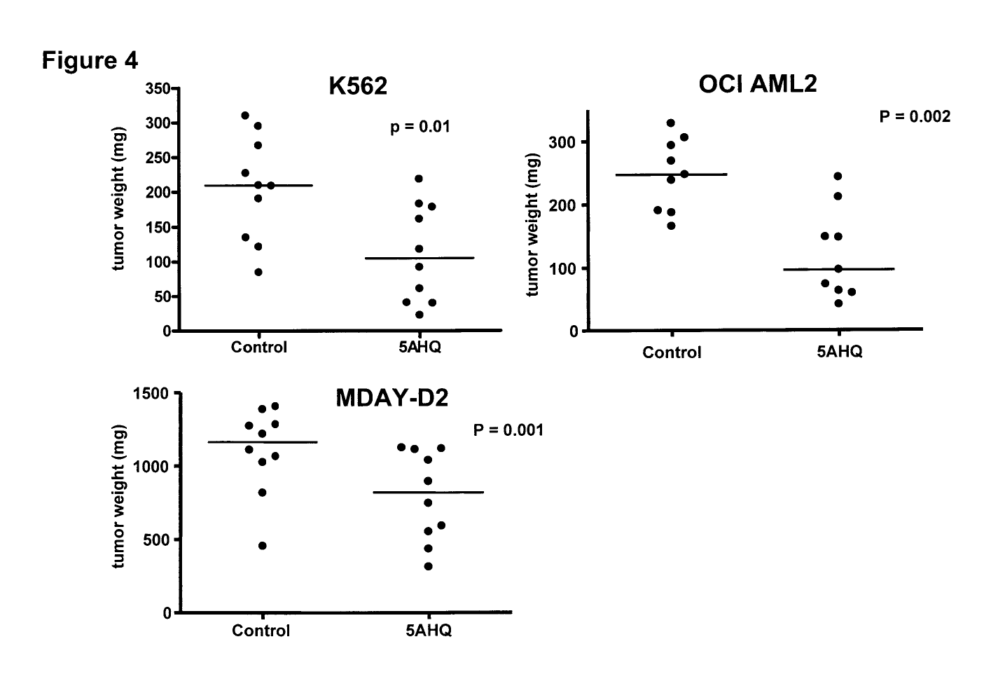

Given the effects on leukemia and lymphoma cell lines and primary patient samples, the compounds were tested in a leukemia/lymphoma xenograft model. MDAY-D2 leukemia/lymphoma cells were injected intraperitoneally into DBA2 mice. Mice were then treated twice daily with oral clioquinol (100 mg/kg) for 10 days. Ten days after treatment, mice were sacrificed and the weight and volume of the intraperitoneal tumor was measured. Oral clioquinol delayed tumor progression without untoward toxicity or reduction in body weight. Similar results were obtained with a K562 leukemia, MDAY-D2 leukemia/lymphoma and OCI AML2 human leukemia xenograft (FIG. 4) K562 leukemia, MDAY-D2 leukemia/lymphoma or OCI AML2 human leukemia cells were injected subcutaneous into sublethally irradiated NOD-Scid mice. Animals were subsequently treated with 5AHQ. The 5AHQ treatment was initiated when tumors reached volumes of 200 mm³ at which time mice were randomized to receive 100 mg/kg of 5AHQ (treated group) or buffer control (untreated group) for 5 of 7 days. Caliper measurements were performed twice weekly to estimate tumor volume and differences compared between treated and untreated groups. Treatment with 5AHQ delayed tumor growth in this mouse model (FIG. 4).

### Compounds of the Application Display Anti-Leukemia/Anti-Lymphoma Activity in Cultured Cells and In Vivo.

LP-1 myeloma cells, MDAY-D2 murine leukemia/lymphoma cells, and OCI-AML2 human leukemia cells and A549 lung cancer cells were treated with increasing concentrations of compounds 5AHQ, HNQ, BCQ, COQ, and DiiodoQ (Table 1). 72 hours after incubation, cell viability was determined by an MTS assay. The compounds of the induced cell death of the tested leukemia and myeloma cell lines with an LD₅₀ in the low micromolar range. In contrast, 5AHQ and HNQ were less cytotoxic to solid tumor cell lines with an LD50 approximately 2-3 fold higher. Given the effects on cultured cells, 5AHQ was evaluated in a mouse model of leukemia. K562 cells were injected subcutaneously into NOD SCID mice. One week after tumors had formed, mice were treated with 5AHQ (50 mg/kg) in water or water alone by oral gavage. Three weeks after injection of cells, the mice were sacrificed, the tumors excised and weighed. 5AHQ reduced tumor weight and volume (FIG. 4). No significant toxicity from 5AHQ was observed.

TABLE 1

| | | | | | IC₅₀ | | |
|---|---|---|---|---|---|---|---|
| | | | LD₅₀ (μm) | | (μM) | K | |
| Name | Structure | Physical Data | whole cell | | 20S | (stability) | Cu:L |
| 5AHQ | 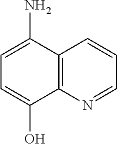 | C9H8N2O 160.175 | LP1 AML2 MDAY-D2 A549 | 12.5 3.9 7.8 31.25 | 3.91 | 9.84 | 1:2 |
| HNQ | 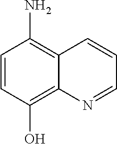 | C9H6N2O3 190.156 | LP1 | 15.6 | 15.6 | 9.13 | 1:2 |
| Clioquinol | 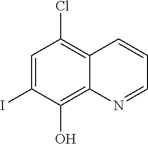 | C9H5ClINO 305.5 | LP1 AML2 MDAY-D2 A549 | 31.25 3.9 15.6 250 | 12.5 | 9.06 | 1:2 |
| BCQ | 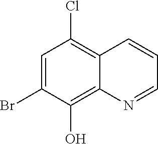 | C9H5BrClNO 258.499 | LP1 | 31.25 | 15.6 | 10.24 | 1:2 |
| COQ | 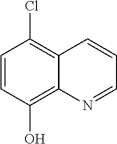 | C9H6ClNO 179.603 | LP1 AML2 MDAY-D2 A549 | 31.25 3.9 3.9 62.5 | 62.5 | 8.89 | 1:2 |
| DiiodoQ | 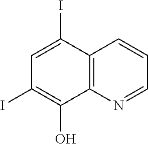 | C9H5I2NO 396.95 | MDAY-D2 AML2 A549 | 31.25 62.5 250 | >1000 | | |

### Discussion

Acute myeloid leukemia (AML), lymphoma and multiple myeloma (MM) are malignant diseases resulting in the proliferation of abnormal cells of myeloid (AML) and lymphoid (lymphoma and myeloma) origin. These diseases are characterized by poor responses to standard therapies. It would be advantageous for these patients and those with relapsed refractory disease if novel therapies were available. As many of these patients are frail, therapies that achieve an anti-myeloma, lymphoma or leukemia effect without significant toxicity are highly desirable.

### Example 2

### Effect of HNQ on Tumor Growth in a Xenograph Animal Model of Leukemia and Lymphoma

SCID mice are injected intraperitoneally with MDAY-D2 murine leukemia cells. Mice are then treated twice daily by oral gavage with HNQ (10 mg/kg or 50 mg/kg) dissolved in intralipid or water, or intralipid control or water control for 10 days. After treatment, 10 mg/kg or 50 mg/kg) dissolved in intralipid or water or intralipid control or water control. Mice are treated daily for 14 or 21 days Three weeks after injection of cells, mice are sacrificed, the subcutaneous tumor excised, and the weight and volume of the tumor is measured.

### Example 3

### Toxicity of 5AHQ

A toxicology protocol for 4 dose levels of 5AHQ given daily X14 days was developed for oral gavage and i.p. administration. Dose levels of 5AHQ for oral toxicology study (300, 200, 100, and 50 mg/kg/day) were administered according to the protocol outlined in the table below. Efficacy dose in mice xenografts was found to be 50 mg/kg/day.

The dose level for i.p administration can be determined according to the protocol outlined in the table below.

| Delivery | Oral gavage (mg/kg) | | i.p. injection | |
|---|---|---|---|---|
| Dosage (mg/kg) | 1. (Control) | 0 | 1. (Control) | 0 |
| | 2. (Low) | 50 | 2. (Low) | 0.1 |
| | 3. (Mid-low) | 100 | 3. (Mid-low) | 0.5 |
| | 4. (Mid-high) | 200 | 4. (Mid-high) | 5 |
| | 5. (High) | 300 | 5. (High) | 10 |

| | |
|---|---|
| Frequency | once daily |
| Treatment | 14 days |
| duration | |
| No. of animals | 5 male/5 female in each group |
| Recovery | 7 days; group 1 and 5; 5 male/5 female in |
| | each group |
| Reporting | morbidity/mortality; clinical signs; body |
| | weight; food consumption; haematology; |
| | toxicokinetics; pharmacokinetics; gross |
| | examination |

### Example 4

### Toxicity of HNQ

A toxicology protocol for 4 dose levels of HNQ given daily X14 days is developed for oral gavage and for i.p. administration.

Dose levels of HNQ for oral toxicology study can include 200, 100, 50, and 10 mg/kg/day. As indicated in the Table below, rodents are administered a dose level of HNQ daily for 14 days and assessed for mortality and other indicators. If no there is no mortality, the dose level of HNQ may be increased and the method repeated.

Similarly, the dose level for i.p administration can be determined according to the protocol outlined in the Table below.

| Delivery | Oral gavage (mg/kg) | | i.p. injection | |
|---|---|---|---|---|
| Dosage (mg/kg) | 1. (Control) | 0 | 1. (Control) | 0 |
| | 2. (Low) | 10 | 2. (Low) | 2 |
| | 3. (Mid-low) | 50 | 3. (Mid-low) | 10 |
| | 4. (Mid-high) | 100 | 4. (Mid-high) | 50 |
| | 5. (High) | 200 | 5. (High) | 100 |

| | |
|---|---|
| Frequency | once daily |
| Treatment | 14 days |
| duration | |
| No. of animals | 5 male/5 female in each group |
| Recovery | 7 days; group 1 and 5, and/or grous 1-5; 5 male/ |
| | 5 female in each group |
| Reporting | morbidity/mortality; clinical signs; body |
| | weight; food consumption; haematology; |
| | toxicokinetics; pharmacokinetics; gross |
| | examination |

The toxicity of BCQ and COQ can be similarly determined.

### Example 5

### Metabolism/Bioanalytical Assay/Stability

Development of a method for bioanalytical assay for 5AHQ levels has been initiated. The level of 5AHQ, dissolved in water or DMSO, is detected in the water or DMSO solution at various time points under various conditions and/or at various time points after administration in plasma using for example NMR and MS. The levels detected under the different conditions are compared to assess 5AHQ levels and metabolism of 5AHQ.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### CITATIONS FOR REFERENCES REFERRED TO IN THE SPECIFICATION

1. Richardson P G, Sonneveld P, Schuster M W, et al. Bortezomib or high-dose dexamethasone for relapsed multiple myeloma. N Engl J Med 2005; 352:2487-98.
2. Goldberg A L. Protein degradation and protection against misfolded or damaged proteins. Nature 2003; 426:895-9.
3. Nalepa G, Rolfe M, and Harper J W. Drug discovery in the ubiquitin-proteasome system. Nat Rev Drug Discov 2006; 5:596-613.
4. Passmore L A and Barford D. Getting into position: the catalytic mechanisms of protein ubiquitylation. Biochem J 2004; 379:513-25.
5. Meister S, Schubert U, Neubert K, et al. Extensive immunoglobulin production sensitizes myeloma cells for proteasome inhibition. Cancer Res 2007; 67:1783-92.
6. Bazzaro M, Lee M K, Zoso A, et al. Ubiquitin-proteasome system stress sensitizes ovarian cancer to proteasome inhibitor-induced apoptosis. Cancer Res 2006; 66:3754-63.
7. Hideshima T, Richardson P, Chauhan D, et al. The proteasome inhibitor PS-341 inhibits growth, induces apoptosis, and overcomes drug resistance in human multiple myeloma cells. Cancer Res 2001; 61:3071-6.
8. Chauhan D, Catley L, Li G, et al. A novel orally active proteasome inhibitor induces apoptosis in multiple myeloma cells with mechanisms distinct from Bortezomib. Cancer Cell 2005; 8:407-19.
9. Arrigo A P, Tanaka K, Goldberg A L, and Welch W J. Identity of the 19S ‘prosome’ particle with the large multifunctional protease complex of mammalian cells (the proteasome). Nature 1988; 331:192-4.
10. Kopp F and Kuehn L. Orientation of the 19S regulator relative to the 20S core proteasome: an immunoelectron microscopic study. J Mol Biol 2003; 329:9-14.
11. Seemuller E, Lupas A, Stock D, Lowe J, Huber R, and Baumeister W. Proteasome from Thermoplasma acidophilum: a threonine protease. Science 1995; 268:579-82.
12. Lowe J, Stock D, Jap B, Zwickl P, Baumeister W, and Huber R. Crystal structure of the 20S proteasome from the archaeon T. acidophilum at 3.4 A resolution. Science 1995; 268:533-9.
13. Kisselev A F, Akopian T N, Castillo V, and Goldberg A L. Proteasome active sites allosterically regulate each other, suggesting a cyclical bite-chew mechanism for protein breakdown. Mol Cell 1999; 4:395-402.
14. Williamson M J, Blank J L, Bruzzese F J, et al. Comparison of biochemical and biological effects of ML858 (salinosporamide A) and bortezomib. Mol Cancer Ther 2006; 5:3052-61.
15. Groll M, Huber R, and Potts B C. Crystal structures of Salinosporamide A (NPI-0052) and B (NPI-0047) in complex with the 20S proteasome reveal important consequences of beta-lactone ring opening and a mechanism for irreversible binding. J Am Chem Soc 2006; 128:5136-41.
16. Groll M, Berkers C R, Ploegh H L, and Ovaa H. Crystal structure of the boronic acid-based proteasome inhibitor bortezomib in complex with the yeast 20S proteasome. Structure 2006; 14:451-6.
17. Yata K, Sadahira Y, Otsuki T, et al. Cell cycle analysis and expression of cell cycle regulator genes in myeloma cells overexpressing cyclin D1. Br J Haematol 2001; 114:591-9.
18. Ely S, Di Liberto M, Niesvizky R, et al. Mutually exclusive cyclin-dependent kinase 4/cyclin D1 and cyclin-dependent kinase 6/cyclin D2 pairing inactivates retinoblastoma protein and promotes cell cycle dysregulation in multiple myeloma. Cancer Res 2005; 65:11345-53.
19. Bergsagel P L, Kuehl W M, Zhan F, Sawyer J, Barlogie B, and Shaughnessy Jr J. Cyclin D dysregulation: an early and unifying pathogenic event in multiple myeloma. Blood 2005.
20. Capel I D, Pinnock M H, Williams D C, and Hanham I W. The serum levels of some trace and bulk elements in cancer patients. Oncology 1982; 39:38-41.
21. Carpentieri U, Myers J, Thorpe L, Daeschner C W, 3rd, and Haggard M E. Copper, zinc, and iron in normal and leukemic lymphocytes from children. Cancer Res 1986; 46:981-4.
22. Zuo X L, Chen J M, Zhou X, Li X Z, and Mei G Y. Levels of selenium, zinc, copper, and antioxidant enzyme activity in patients with leukemia. Biol Trace Elem Res 2006; 114:41-53.
23. Ding W Q, Liu B, Vaught J L, Yamauchi H, and Lind S E. Anticancer activity of the antibiotic clioquinol. Cancer Res 2005; 65:3389-95.
24. Daniel K G, Chen D, Orlu S, Cui Q C, Miller F R, and Dou Q P. Clioquinol and pyrrolidine dithiocarbamate complex with copper to form proteasome inhibitors and apoptosis inducers in human breast cancer cells. Breast Cancer Res 2005; 7:R897-908.
25. Chen D, Cui Q C, Yang H, et al. Clioquinol, a therapeutic agent for Alzheimer's disease, has proteasome-inhibitory, androgen receptor-suppressing, apoptosis-inducing, and antitumor activities in human prostate cancer cells and xenografts. Cancer Res 2007; 67:1636-44.
26. Carter B Z, Gronda M, Wang Z, et al. Small-molecule XIAP inhibitors derepress downstream effector caspases and induce apoptosis of acute myeloid leukemia cells. Blood 2005; 105:4043-50.
27. Hurt E M, Wiestner A, Rosenwald A, et al. Overexpression of c-maf is a frequent oncogenic event in multiple myeloma that promotes proliferation and pathological interactions with bone marrow stroma. Cancer Cell 2004; 5:191-9.
28. Mao X, Stewart A K, Hurren R, et al. A chemical biology screen identifies glucocorticoids that regulate c-maf expression by increasing its proteasomal degradation through upregulation of ubiquitin. Blood 2007; 110:4047-5054.
29. Chemy R A, Atwood C S, Xilinas M E, et al. Treatment with a copper-zinc chelator markedly and rapidly inhibits beta-amyloid accumulation in Alzheimer's disease transgenic mice. Neuron 2001; 30:665-76.
30. Jaffe E S, Harris N L, Stein H, Isaacson P G. Classification of lymphoid neoplasms: the microscope as a tool for disease discovery. Blood. 2008; 112:4384-4399.
31. Mao X, Li X, Sprangers R, et al. Clioquinol inhibits the proteasome and displays preclinical activity in leukemia and myeloma. Leukemia. 2008.
32. Mao X, Liang S B, Hurren R, et al. Cyproheptadine displays preclinical activity in myeloma and leukemia. Blood. 2008; 112:760-769.

1. A method of treating a proliferative disease involving increased D-cyclin and/or a hematological malignancy comprising administering to a subject in need thereof, an effective amount of one or more compounds selected from a compound of Formula I, and pharmaceutically acceptable salts, solvates and prodrugs thereof: 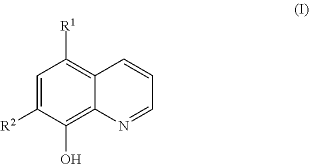
wherein
R¹ is selected from NO₂, NH₂, NH(C₁₋₆alkyl) and N(C₁₋₆alkyl)(C₁₋₆alkyl);
R² is selected from H, halogen, C₁₋₆alkyl, and fluoro-substituted C₁₋₆alkyl, or
R¹ is Cl and R² is Br or H.

2. The method of claim 1 for treating a hematological malignancy comprising administering to subject in need thereof, an effective amount of a compound selected from one or more compounds of Formula I as defined in claim 1 and pharmaceutically acceptable salts, solvates and prodrugs thereof.

3. The method according to claim 1, wherein said hematological malignancy is multiple myeloma, leukemia or lymphoma.

4. (canceled)

5. The method according to claim 3, wherein said leukemia is selected from acute myeloid leukemia and acute lymphocytic leukemia.

6. (canceled)

7. The method according to claim 3 wherein said lymphoma is non-Hodgkin's lymphoma.

8. The method according to claim 7 wherein the non-Hodgkin's lymphoma is a low grade non-Hodgkin's lymphoma, or a high grade non-Hodgkins lymphoma.

9. (canceled)

10. The method according to claim 3 wherein said lymphoma is Hodgkin's lymphoma.

11. The method according to claim 1, wherein, in the compound of Formula I, R¹ is selected from NO₂, NH₂, NH(C₁₋₄ alkyl) and N(C₁₋₄ alkyl)(C₁₋₄ alkyl).

12. The method according to claim 1, wherein, the compound of Formula I R¹ is selected from NO₂, NH₂, NHCH₃ and N(CH₃)₂.

13. (canceled)

14. The method according to claim 1, wherein in the compound of Formula I R₂ is selected from H, Cl, F, Br, I, C₁₋₄ alkyl and fluoro-substituted C₁₋₄ alkyl.

15. The method according to claim 1, wherein, in the compound of Formula I R₂ is selected from H, Cl, I, Br, CH₃ and CF₃.

16. (canceled)

17. The method according to claim 1, wherein, in the compound of Formula I, R¹ is selected from NO₂, NH₂ and R² is selected from H and halogen.

18. The method according to claim 1 wherein the one or more compounds of Formula I are selected from 5-amino-8-hydroxyquinoline (AHQ), 8-hydroxy-5-nitroquinoline (HNQ), 7-bromo-5-chloro-8-hydroxyquinoline (BCQ) and 5-chloro-8-hydroxyquinoline (COQ), and pharmaceutically acceptable salts, solvates and prodrugs thereof.

19-22. (canceled)

23. The method according to claim 1, wherein said effective amount is about 1 to about 200 mg/kg body weight.

24. (canceled)

25. The method according to claim 1, wherein the effective amount is about 20 to about 5000 mg, about 100 to about 1500 mg, or about 400 to about 1200 mg.

26-36. (canceled)

37. A pharmaceutical composition comprising a compound selected from one or more compounds of Formula I as defined in claim 1, and pharmaceutically acceptable salts, solvates and prodrugs thereof, and a pharmaceutically acceptable carrier in a solid dosage form, a liquid dosage form, or an injectable dosage form that comprises about 10 to about 5000 mg, about 10 to about 1500 mg, or about 30 to about 300 mg of the compound.

38-40. (canceled)

41. A pharmaceutical composition for treatment of acute myeloid leukemia or multiple myeloma in a subject, which composition comprises as active ingredient a compound selected from a compound of Formula I as defined in claim 1, and a pharmaceutically acceptable salt, solvate and prodrug thereof, and a pharmaceutically acceptable carrier in unit dosage form.

42. The pharmaceutical composition of claim 41, wherein the dosage form is for oral administration or for injection.

43. (canceled)

44. A pharmaceutical composition comprising a compound selected from a compound of Formula I as defined in claim 1, and a pharmaceutically acceptable salt, solvate and prodrug thereof, and a pharmaceutically acceptable carrier in unit dosage form in an amount to provide about 1 to about 200 mg, about 2 to about 100 mg, or about 5 to about 50 mg of the compound per kg body weight formulated into a solid oral dosage form, a liquid dosage form, or an injectable dosage form.

45. (canceled)

46. The composition as claimed according to claim 1 useful for the treatment of a hematological malignancy, the hematological malignancy selected from the group comprising leukemia and multiple myeloma.

47. The composition according to claim 46 wherein the leukemia is acute myeloid leukemia or acute lymphocytic leukemia.
